(19) United States
(12) Patent Application Publication
(10) Pub. No.: US 2017/0281294 A1
(43) Pub. Date: Oct. 5, 2017

(54) MARKING METHOD AND COMPUTERIZED TOMOGRAPHY APPARATUS

(71) Applicant: General Electric Company, Schenectady, NY (US)

(72) Inventors: Haifeng WU, Beijing (CN); Ying WANG, Beijing (CN)

(21) Appl. No.: 15/474,025

(22) Filed: Mar. 30, 2017

(30) Foreign Application Priority Data
Mar. 31, 2016 (CN) 201610196959.6

(51) Int. Cl. A61B 90/00; A61B 6/04; A61B 6/03

(52) U.S. Cl. CPC A61B 90/39; A61B 6/032; A61B 6/0407; A61B 2090/395; A61B 2090/3983

(57) ABSTRACT

The present invention provides a marking method and a computerized tomography apparatus, the method comprising: scanning a target object to obtain an image of the target object; determining an intervention position on a surface of the target object according to a position of a part of interest in the image of the target object; marking the intervention position on the surface of the target object. Therefore, the intervention position may be easily marked on the surface of the target object.

### BACKGROUND TO THE INVENTION

The present invention relates to a marking method and a computerized tomography apparatus.

In recent years, an intervention method guided by the way of computerized tomography (CT) imaging has been widely used. Usually, an operator (e.g., a doctor) performs CT scan on a target object (e.g., a patient) to obtain a CT image of the target object. Then, the operator guides insertion of an intervention tool (e.g., a needle) in real time according to the obtained CT image. For example, the operator needs to formulate an intervention point of the intervention tool on a surface of the target object, an angle of the intervention tool, and an insertion path of the intervention tool within the target object, etc. according to the CT image. However, since the target object is still on a bracket (bed) of a CT apparatus at this moment and ISO of the CT apparatus is within the target object, it is difficult to mark the intervention point and the intervention angle on a body of the target object.

### BRIEF DESCRIPTION OF THE INVENTION

An objective of exemplary embodiments of the present invention is to overcome the aforementioned and/or other problems in the prior art. Therefore, the exemplary embodiments of the present invention provide a marking method and a computerized tomography apparatus that may easily mark an intervention position on a surface of a target object.

According to an exemplary embodiment, a marking method may comprise: scanning a target object to obtain an image of the target object; determining an intervention position on a surface of the target object according to a position of a part of interest in the image of the target object; marking the intervention position on the surface of the target object.

According to another exemplary embodiment, a computerized tomography apparatus may comprise: a target-object-bracket configured to place a target object to be scanned; an X-ray scanning device configured to scan the target object to obtain an image of the target object; a processing device configured to determine an intervention position on a surface of the target object according to a position of a part of interest in the image of the target object obtained by the X-ray scanning device; a marking device configured to mark the determined intervention position.

Other features and aspects will be apparent through the following detailed description, figures and claims.

### BRIEF DESCRIPTION OF THE DRAWINGS

The present invention can be understood better in light of the description of exemplary embodiments of the present invention with reference to the accompanying drawings, in which:

FIG. 1 is a schematic diagram of a CT apparatus according to an exemplary embodiment;

FIG. 2 and FIG. 3 are diagrams for illustrating an operation of determining an intervention position on a surface of a target object according to exemplary embodiments;

FIG. 4 is a flow chart of a marking method according to an exemplary embodiment.

### DETAILED DESCRIPTION OF THE INVENTION

Hereafter, a detailed description will be given for preferred embodiments of the present disclosure. It should be pointed out that in the detailed description of the embodiments, for simplicity and conciseness, it is impossible for the Description to describe all the features of the practical embodiments in details. It should be understood that in the process of a practical implementation of any embodiment, just as in the process of an engineering project or a designing project, in order to achieve a specific goal of the developer and in order to satisfy some system-related or business-related constraints, a variety of decisions will usually be made, which will also be varied from one embodiment to another. In addition, it can also be understood that although the effort made in such developing process may be complex and time-consuming, some variations such as design, manufacture and production on the basis of the technical contents disclosed in the disclosure are just customary technical means in the art for those of ordinary skilled in the art associated with the contents disclosed in the present disclosure, which should not be regarded as insufficient disclosure of the present disclosure.

Unless defined otherwise, all the technical or scientific terms used in the Claims and the Description should have the same meanings as commonly understood by one of ordinary skilled in the art to which the present disclosure belongs. The terms “first”, “second” and the like in the Description and the Claims of the present utility model do not mean any sequential order, number or importance, but are only used for distinguishing different components. The terms “a”, “an” and the like do not denote a limitation of quantity, but denote the existence of at least one. The terms “comprises”, “comprising”, “includes”, “including” and the like mean that the element or object in front of the “comprises”, “comprising”, “includes” and “including” covers the elements or objects and their equivalents illustrated following the “comprises”, “comprising”, “includes” and “including”, but do not exclude other elements or objects. The term “coupled” or “connected” or the like is not limited to being connected physically or mechanically, nor limited to being connected directly or indirectly.

FIG. 1 is a schematic diagram of a CT apparatus according to an exemplary embodiment.

As shown in FIG. 1, the CT apparatus according to the exemplary embodiment includes a bracket (bed) 100, an X-ray scanning device 300, a processing device 500, and a marking device 700.

The bracket (bed) 100 may place a target object to be scanned, e.g., a patient to be scanned. The bracket (bed) 100 may move to vary a position of the target object placed on the bracket (bed) 100 with respect to the X-ray scanning device 300. For example, the bracket (bed) 100 may move the target object to a position scanned by the X-ray scanning device 300.

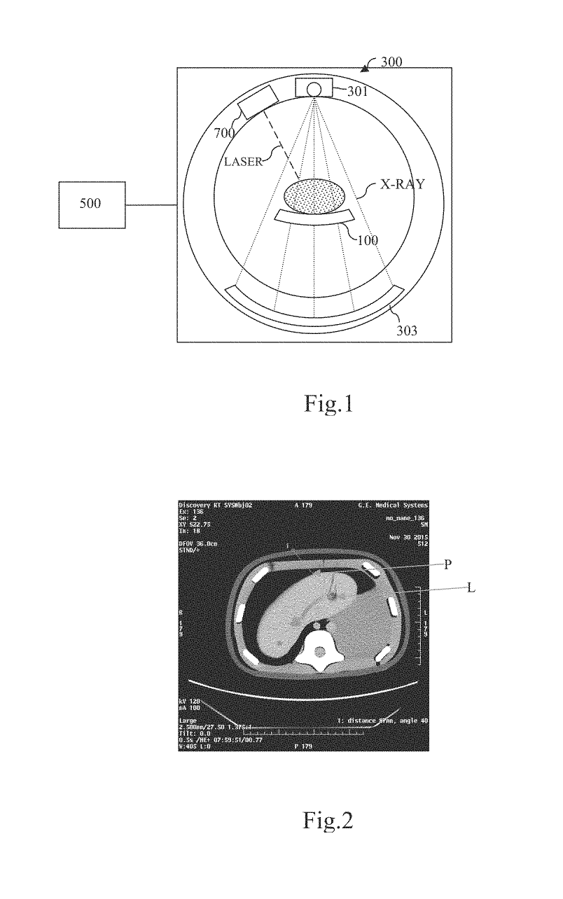

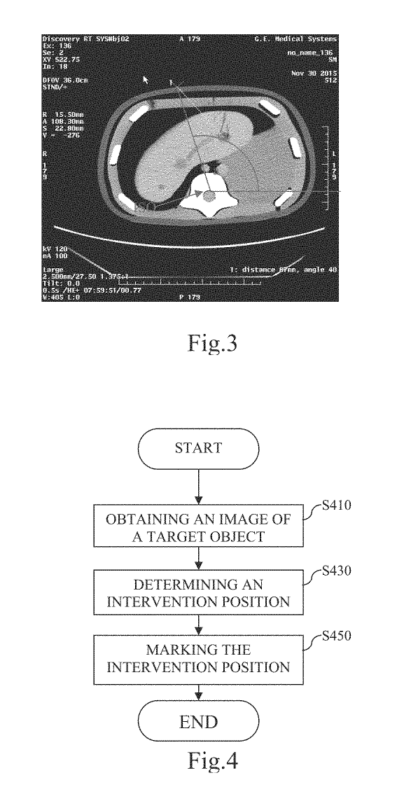

The X-ray scanning device 300 may include an X-ray emitting device 301 and an X-ray receiving device 303. The X-ray emitting device 301 may emit an X-ray. The X-ray receiving device 303 may receive the X-ray that passes through the target object, and may convert the received X-ray into an electric signal. Moreover, although not shown in the figure, the X-ray scanning device 300 may further include an image processing device. The image processing device may receive and process the electric signal converted by the X-ray receiving device 303 to obtain an image of the target object, as shown in FIG. 2 and FIG. 3.

The processing device 500 may receive the image of the target object obtained by the X-ray scanning device 300, and may determine an intervention position on a surface of the target object according to a part of interest in the image. An operation of determining the intervention position on the surface of the target object will be described in details below with reference to FIG. 2 and FIG. 3.

FIG. 2 and FIG. 3 are diagrams for illustrating an operation of determining an intervention position on a surface of a target object according to exemplary embodiments.

As shown in FIG. 2, firstly, an operator (e.g., a doctor) may browse images from axially (Z) scanned images to select an image at which a target is located, i.e., an image containing a part of interest L as shown in FIG. 2, and then the part of interest L is selected in the image of the target object. The part of interest L may be a part of a patient that may be injured. To this end, the CT apparatus according to the exemplary embodiment may include a display device (not shown) and an input device (not shown). The display device may display the image of the target object. The doctor may observe the image displayed by the display device, and may select the part of interest L in the image by operating the input device. Optionally, the part of interest L may be automatically identified and determined by performing a known image identification method by the processing device 500.

Then, the operator may set an intervention path P according to a position of the part of interest in the image of the target object, e.g., using the input device, as shown in FIG. 2. However, the exemplary embodiments are not limited thereto. In other exemplary embodiments, the processing device 500 may automatically set the intervention path P according to pre-defined conditions.

Next, the processing device 500 may determine an intersection point 1 of the intervention path P with the surface of the target object, and thus may determine the intervention position on the surface of the target object according to a position of the intersection point 1 in the image (e.g., the intersection point 1 being located at the second quadrant, which has a coordinate of x=−15.5 mm, y=108.3 mm) and position information of the image with respect to the target object, etc.

As shown in FIG. 3, in one exemplary embodiment, the processing device 500 may calculate an angle from the intersection point 1 to an ISO center of the CT apparatus. In the exemplary embodiment as shown in FIG. 3, the angle “a” from the intersection point 1 to the ISO center of the CT apparatus is:

arctan(x/y)+90°=arctan(15.5/108.3)+90°=98.2°.

Furthermore, the processing device 500 may determine a distance of the intersection point 1 from a Z position of the CT apparatus. For example, as shown in FIG. 3, the processing device 500 may determine that the distance of the intersection point 1 from the Z position of the CT apparatus is +22.8 mm according to the position information of the image with respect to the target object. For example, the operator (e.g., the doctor) may scan a series of images near the target (Z) by the CT apparatus. Then, the doctor may browse the series of images to find the image at which a center of the target is located there from, and may read a coordinate, e.g., 22.75 (≈22.8) directly from a CT image, as shown in FIG. 3. After the intervention position has been determined according to the position of the intersection point 1 in the image and the position information of the image with respect to the target object, the marking device 700 may mark the determined intervention position. For example, the marking device 700 may move according to the calculated angle and the determined distance to form a mark indicating the intervention position on the surface of the target object by the marking device. Optionally, the bracket (bed) 100 supporting the target object may be moved, or both the marking device 700 and the bracket (bed) 100 supporting the target object may be moved, so as to form the mark indicating the intervention position on the surface of the target object.

Herein, the marking device 700 may be installed on a rack of the CT apparatus together with the X-ray scanning device. The marking device 700 is a laser indicating device, which consists of two crossed fan-shaped laser beams, with one passing through a CT scanning plane and the other being perpendicular to the CT scanning plane. The marking device 700 may rotate along a 360-degree scanning plane, and may form crossed laser indicating points on the surface of the target object. Optionally, the marking device 700 may also be a single-beam laser, and may rotate along the 360-degree scanning plane to directly form a laser indicating point on the surface of the target object. After the intervention position has been marked using the marking device 700, the operator (e.g., the doctor) may perform an intervention operation along the intervention path from the intervention position with the intervention tool such as a needle, etc.

The CT apparatus according to the exemplary embodiment may determine the intervention position on the surface of the target object according to the image obtained by the way of CT imaging, and may mark the intervention position using the marking device. Therefore, when the target object is still supported by the bracket (bed) of the CT apparatus and/or at a scanning position in the rack of the CT apparatus, the intervention position may be easily marked on the surface of the patient's body.

FIG. 4 is a flow chart of a marking method according to an exemplary embodiment. As shown in FIG. 4, firstly, a target object may be scanned to obtain an image of the target object (S410). Herein, the target object may be imaged using the computerized tomography apparatus according to the exemplary embodiment as described above to obtain the image of the target object, as shown in FIG. 2 and FIG. 3.

Then, an intervention position on a surface of the target object may be determined according to a position of a part of interest in the image of the target object (S430). As described above with reference to FIG. 2, an intervention path may be set according to the position of the part of interest in the image of the target object, and the intervention position as an intersection point of the intervention path with the surface of the target object may be determined.

Specifically, the operator may observe the image of the target object displayed on a display device of the CT apparatus to determine a part of interest that may be a part of the target object that may be injured, and may select the determined part of interest in the image by an input device. Optionally, the part of interest may be automatically identified and determined by performing a known image identification method by a processing device (500) of the CT apparatus.

Then, the operator may set the intervention path according to the position of the part of interest in the image of the target object, e.g., using the input device, as shown in FIG. 2. However, the exemplary embodiments are not limited thereto. In other exemplary embodiments, the intervention path may be automatically set according to pre-defined conditions by the processing device (500).

Next, an intersection point 1 of an intervention path P with the surface of the target object may be determined, e.g., by the processing device (500), and thus the intervention position on the surface of the target object may be determined according to a position of the intersection point 1 in the image (e.g., a coordinate of the intersection point 1) and position information of the image with respect to the target object, etc. For example, an angle from the intersection point to an ISO center of the CT apparatus may be calculated and a distance of the intersection point from a Z position of the CT apparatus may be determined by the processing device (500).

Finally, the intervention position on the surface of the target object may be marked (S450). For example, at least one of a marking device and a target-object-bracket of the CT apparatus may be moved according to the calculated angle and the determined distance, so as to form a mark indicating the intervention position on the surface of the target object by the marking device.

According to the exemplary embodiments, the intervention position on the surface of the target object may be determined according to the image obtained by the way of CT imaging, and the intervention position may be marked by the marking device of the CT apparatus. Therefore, a process of marking the intervention position may be simplified. Moreover, the intervention position may be marked on the surface of the patient's body when the target object is still supported by the bracket (bed) of the CT apparatus and/or at a scanning position in a rack of the CT apparatus.

Some exemplary embodiments have been described in the above. However, it should be understood that various modifications may be made thereto. For example, if the described techniques are carried out in different orders, and/or if the components in the described system, architecture, apparatus or circuit are combined in different ways and/or replaced or supplemented by additional components or equivalents thereof, proper results may still be achieved. Accordingly, other implementation also falls within a protection range of the Claims.

What is claimed is:

1. A marking method, comprising:
scanning a target object to obtain an image of the target object;
determining an intervention position on a surface of the target object according to a position of a part of interest in the image of the target object;
marking the intervention position on the surface of the target object.

2. The method according to claim 1, wherein the step of obtaining an image of the target object comprises:
imaging the target object using a computerized tomography apparatus to obtain the image of the target object.

3. The method according to claim 1, wherein the step of determining an intervention position on a surface of the target object comprises:
setting an intervention path according to the position of the part of interest in the image of the target object;
determining the intervention position as an intersection point of the intervention path with the surface of the target object.

4. The method according to claim 3, wherein the step of determining the intervention position as an intersection point of the intervention path with the surface of the target object comprises:
calculating an angle from the intersection point to an ISO center of the computerized tomography apparatus;
determining a distance of the intersection point from a Z position of the computerized tomography apparatus.

5. The method according to claim 4, wherein at least one of a marking device and a target-object-bracket of the computerized tomography apparatus is moved according to the calculated angle and the determined distance, so as to form a mark indicating the intervention position on the surface of the target object by the marking device.

6. A computerized tomography apparatus, comprising:
a target-object-bracket configured to place a target object to be scanned;
an X-ray scanning device configured to scan the target object to obtain an image of the target object;
a processing device configured to determine an intervention position on a surface of the target object according to a position of a part of interest in the image of the target object obtained by the X-ray scanning device;
a marking device configured to mark the determined intervention position.

7. The computerized tomography apparatus according to claim 6, wherein the processing device is configured to determine the intervention position as an intersection point of an intervention path set according to the position of the part of interest in the image of the target object with the surface of the target object according to the position of the part of interest and the intervention path.

8. The computerized tomography apparatus according to claim 7, wherein the processing device is configured to calculate an angle from the intersection position to an ISO center of the computerized tomography apparatus and to determine a distance of the intersection position from a Z position of the computerized tomography apparatus.

9. The computerized tomography apparatus according to claim 8, wherein at least one of the marking device and the target object is moved according to the calculated angle and the determined distance, so as to form a mark indicating the intervention position on the surface of the target object by the marking device.
